SEQUENCE LISTING (2 sequences)

SEQ ID NO: 1 - DNA, 498 bp, Homo sapiens
atggtcaaccccaccgtgttcttcgacattgccgtcgacggcgagcccttgggccgcgtctcctttgagctgtttgcagacaaggtcccaaagacagcagaaaattttcgtgctctgagcactggagagaaaggatttggttataagggttcctgctttcacagaattattccagggtttatgtgtcagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggttggatggcaagcatgtggtgtttggcaaagtgaaagaaggcatgaatattgtggaggccatggagcgctttgggtccaggaatggcaagaccagcaagaagatcaccattgctgactgtggacaactcgaataa

SEQ ID NO: 2 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

(19) United States
(12) Patent Application Publication
(10) Pub. No.: US 2010/0203510 A1
(43) Pub. Date: Aug. 12, 2010

(54) PPIA MARKER FOR DIAGNOSIS OF LIVER CANCER AND ANTIBODY, AND SCREENING METHOD OF COMPOUNDS USEFUL FOR INHIBITING LIVER CANCER

(75) Inventors: Dae-Ghon Kim, Jeonju-si (KR); Gyung-Ran Yu, Jeonju-si (KR); Eun-Young Song, Seoul (KR); Hee-Gu Lee, Daejeon (KR); Young-Il Yeom, Daejeon (KR); Jung-Il Lee, Daejeon (KR); Jae-Wha Kim, Daejeon (KR); Na-Young Ji, Hwaseong-si (KR)

Correspondence Address: THE NATH LAW GROUP, 112 South West Street, Alexandria, VA, 22314, US

(73) Assignees: Industrial Cooperation Foundation Chonbuk National University, Jeonju-si (KR); Korea Research Institute Of Bioscience And Biotechnology, Daejeon (KR)

(21) Appl. No.: 12/312,811

(22) PCT Filed: Feb. 10, 2009
(86) PCT No.: PCT/KR2009/000614

§ 371 Date: Jun. 26, 2009

(51) Int. Cl. C12Q 1/68; C07H 21/00; C07K 16/00; C07K 14/00; C12N 15/74; C12N 5/07

(52) U.S. Cl. 435/6; 536/23.1; 536/23.5; 530/387.1; 530/350; 435/320.1; 435/348

(57) ABSTRACT

Disclosed is a marker for diagnosis of liver cancer comprising polynucleotide useful for diagnosis of liver cancer. The marker for liver cancer diagnosis comprises at least one polynucleotide selected from a group consisting of the following polynucleotides (a) to (d):(a) polynucleotide having a base sequence defined by Seq. No. 1 or substantially similar sequence to Seq. No. 1; (b) polynucleotide encoding specific protein composed of an amino acid sequence defined by Seq. No. 2; (c) polynucleotide that comprises Seq. No. 2, in which at least one amino acid is substituted, deleted, inserted and/or added, and that encodes protein functionally equivalent to specific protein composed of Seq. No. 2; and (d) polynucleotide that is encoded by another polynucleotide hybridized with polynucleotide having Seq. No. 1 under stringent conditions, and that encodes protein functionally equivalent to specific protein composed of Seq. No. 2.

### TECHNICAL FIELD

The present invention relates to technical processes for using PPIA encoding polynucleotide, modified sequences thereof and/or PPIA proteins expressed by the same, as markers for diagnosis and/or prognosis of liver cancer, more particularly, to PPIA marker for diagnosis of liver cancer, antibody, and a screening method of compounds useful for treatment and/or inhibition of liver cancer.

### BACKGROUND ART

For diagnosis of hepatocellular carcinoma (often called hepatoma), there have been commonly used various examinations including, for example, imaging diagnosis studies such as ultrasonography, computer tomography (CT) or magnetic resonance imaging (MRI) test, blood studies or serum screening such as AFP or PIVKA-II, and pathologic tissue studies using tissue biopsy.

In case of AFP assay, about less than 70 percent of patients with hepatoma show higher AFP assay value. However, patients with chronic liver diseases also often demonstrate higher AFP assay value and, therefore, it is absolutely necessary to differentially diagnose or distinguish hepatoma patient from patients with the other liver diseases. For patients with early hepatocellular carcinoma, they often show lowered AFP assay value. In PIVKA-II (protein induced by vitamin K antagonist-II) assay, positive rate is less than 50 percent but specificity to hepatocellular carcinoma is relatively higher.

Based on this reason, it is known that diagnosis accuracy can be improved by application of the above assays in combination thereof. However, there is a requirement for development of more specific cancer makers to characterize or identify positive and/or negative cases in regard to hepatocellular carcinoma.

Pathologic tissue studies using tissue biopsy are important to accurately diagnose liver diseases. But, application of pathologic characteristics alone is sometimes insufficient to differentially recognize cancer tissues, especially, early hepatocellular carcinoma tissues from other non-cancer tissues. For instance, large regenerative nodules or early well-differentiated hepatocellular carcinoma are sometimes in the form of minor lesions. Since amount of samples may be limited when tumor tissue procurement is carried out by means of percutaneous needle aspiration biopsy assay, there is still a requirement for more reliable diagnosis techniques. Consequently, it is preferable to develop cancer-specific antibodies useful for differentially recognizing early hepatocellular carcinoma tissues from other non-cancer tissues.

A variety of cancer therapies, in particular, hepatocellular carcinoma treatments are generally known, which include surgical excision, transcatheter arterial (chemo) embolization (TACE), percutaneous ethanol injection, microwave coagulation therapy, etc.

Some medical institutions prefer to apply the most confident treatment in their own facilities, rather than optimum treatments for individual cases and, at present, there exists no clear and reliable criteria for selection of diagnosis reference and/or remedy prescriptions.

A plurality of gene groups causing abnormal conditions of liver cancer are useful in diagnosis thereof by clustering the gene groups. Although genes well known to cause abnormal condition in relation to hepatocellular carcinoma include, for example, IGF-II, c-myc, cyclin D or VEGF, etc., genomic abnormality related to generation and progress of hepatocellular carcinoma is not yet clearly disclosed.

PPIA, also referred to as cyclophilin, CypA belongs to immunophilin families and has been identified from intercellular receptor of cyclosporine A. It was reported in prior art that PPIA initially exists in cells and is secreted by inflammable stimulation (see B. Sherry et al., Proc Natl Acad Sci USA 89 (1992), pp. 3511-3515). Secreted PPIA has various features such as chemical sensitivity, cellular signals, etc. but, there is not still clearly disclosed how the secreted PPIA is expressed in live cancer cells.

### DISCLOSURE

### Technical Problem

Accordingly, the present invention is directed to solve the problems described above in regard to conventional methods and verifies application of PPIA in diagnosis and treatment of liver cancer and utility of PPIA studies in relation to carcinogenesis.

Therefore, an object of the present invention is to provide a marker for diagnosis of liver cancer, antibody and a diagnosis kit for liver cancer comprising the marker and/or the antibody based on the above verified result.

Another object of the present invention is to provide a screening method of compounds useful for treatment of liver cancer by using the marker and/or the antibody described above.

### Technical Solution

In order to accomplish the above objects, the present invention provides (1) a marker for liver cancer diagnosis comprising any one of polynucleotides as defined in the following (a) to (d):(a) polynucleotide having a base sequence defined by Seq. No. 1 or substantially similar sequence to Seq. No. 1; (b) polynucleotide encoding specific protein composed of an amino acid sequence defined by Seq. No. 2; (c) polynucleotide that comprises Seq. No. 2, in which at least one amino acid is substituted, deleted, inserted and/or added, and that encodes protein functionally equivalent to specific protein composed of Seq. No. 2; and (d) polynucleotide that is encoded by another polynucleotide hybridized with polynucleotide having Seq. No. 1 under stringent conditions, and that encodes protein functionally equivalent to specific protein composed of Seq. No. 2.

(2) In the present invention, the marker further comprises alternative polynucleotide encoding partial peptide of protein encoded by the polynucleotide described in above (1).

(3) In the present invention, the marker further comprises protein encoded by the polynucleotide described in above (1) and/or partial peptide of the protein.

(4) The present invention further provides an antibody for diagnosis of liver cancer comprising polynucleotide defined in above (1) and/or protein defined in above (3) as antigen, which is able to be conjugated to the polynucleotide and/or the protein.

(5) The present invention further provides a liver cancer diagnosis kit including polynucleotide defined in above (1) and/or protein defined in above (3) as a marker for diagnosis of liver cancer.

(6) The present invention further provides a liver cancer diagnosis kit including antigen defined in above (4).

(7) The present invention further provides a screening method of compounds for control expression of polynucleotide defined in above (1) and/or protein defined in above (3), comprising the steps of: (a) contacting candidate compounds with cells; (b) comparing gene expression level of the polynucleotide defined in above (1) and/or the protein defined in above (3) in the cells to that of a control; and (c) selecting the candidate compound that alters gene expression level.

(8) The present invention further provides a diagnosis method of liver cancer, comprising the steps of (a) measuring content of polynucleotide defined in above (1) and/or protein defined in above (3) in a biota sample; and (b) comparing the measured value to that of a control, in order to detect liver cancer.

### ADVANTAGEOUS EFFECTS

According to the present invention, amount of PPIA secretion in patients suffering from liver cancer is larger than that in normal persons and, based on this discovery, PPIA is useful for diagnosis and treatment of liver cancer and PPIA studies, and therefore may be widely applied to manufacturing of diagnostics and/or therapeutics.

### DESCRIPTION OF DRAWINGS

The above objects, features and advantages of the present invention will become more apparent to those skilled in the related art in conjunction with the accompanying drawings. In the drawings:

FIG. 1 is photographs showing degree of expressing PPIA in liver cancer tissues: the upper photograph shows a comparison of PPIA expressions between differentiated cancer tissues and non-differentiated tissues of a patient suffering from liver cancer by means of Northern blot assay, and the lower photograph shows a result of PPIA expressions in differentiation stages of liver cancer;

FIG. 2 shows a result of PPIA protein expression by means of Western blot assay using PPIA polyclonal antibody;

FIG. 3 is a photograph showing degree of PPIA expression in supernatant of culture medium for liver cancer cell line;

FIG. 4 is photographs showing expression of PPIA protein by immunohistochemical assay;

FIG. 5 is bar graphs showing difference of PPIA secretions by immunodot assay; and

FIG. 6 is line graphs showing dilution of a liver cancer sample and a normal sample by ELISA assay.

### BEST MODE

Hereinafter, the present invention will be described in detail from the following description.

An aspect of the present invention in order to accomplish the above objects is to provide a marker for liver cancer diagnosis comprising any one of polynucleotides as defined in the following (a) to (d):(a) polynucleotide having a base sequence defined by Seq. No. 1 or substantially the same sequence to Seq. No. 1; (b) polynucleotide encoding specific protein composed of an amino acid sequence defined by Seq. No. 2; (c) polynucleotide that comprises Seq. No. 2, in which at least one amino acid is substituted, deleted, inserted and/or added, and that encodes protein functionally equivalent to specific protein composed of Seq. No. 2; and (d) polynucleotide that is encoded by another polynucleotide hybridized with polynucleotide having Seq. No. 1 under stringent conditions, and that encodes protein functionally equivalent to specific protein composed of Seq. No. 2.

“Substantially the same base sequence to” a base sequence represented by Seq. No. 1 according to the present invention means a base sequence having sequence homology of at least 70%, preferably at least 80%, more preferably at least 90%, particularly preferably at least 95%, and most preferably at least 98% to Seq. No. 1, which includes a base sequence encoding protein with the same function as of specific encoding protein among Seq. No. 1.

Polynucleotide according to the present invention may comprise DNA or RNA and, preferably, mRNA.

At least one amino acid in Seq. No. 2 comprises specifically substituted, deleted, inserted and/or added amino acid sequence. Polynucleotide encoding protein functionally equivalent to specific protein composed of amino acid sequence (that is, Seq. No. 2) may comprise protein with substantially the same function to the specific protein composed of Seq. No. 2, wherein amino acid is substituted by another one having similar chemical characteristics without inhibiting essential functions of protein such as substitution of leucine with isoleucine, and/or both terminals of a protein are partially deleted by translation and regulation thereof.

Polynucleotide hybridized with polynucleotide composed of Seq. No. 1 under stringent conditions comprises, for example, polynucleotide including another base sequence having sequence homology of at least 50%, preferably at least 60%, more preferably at least 70%, particularly preferably at least 80%, and most preferably at least 90% to complementary sequence of Seq. No. 1. Hybridization of the polynucleotide may be conducted by conventionally known processes in the related art, for example, a method disclosed in Molecular Cloning, Second edition, J. Sambrook et al., Cold Spring Harbor Lab. Press, 1989, the entire contents of which are hereby incorporated by reference into the present invention. Alternatively, hybridization using a library commercially available in the market is performed according to instructions supplied with the library. Such hybridization is preferably carried out under stringent conditions including, for example, sodium concentration of about 19 to 40 mM, preferably, 19 to 20 mM and/or temperature of about 50 to 70° C., preferably, 60 to 65° C. More preferably, concentration of sodium is about 19 mM and temperature is 65° C.

The present invention further provides a marker for diagnosis of liver cancer, which comprises polynucleotide encoding partial peptide of specific protein encoded by the polynucleotide described above. Such partial peptide includes, for example, peptide containing amino acid sequence having sequence homology of at least 50%, preferably at least 60%, more preferably at least 70%, particularly preferably at least 80%, and most preferably at least 90% to Seq. No. 2.

The present invention further provides antibodies for diagnosis of liver cancer, which can be combined with the above mentioned polynucleotide or protein as antigens.

The antibodies of the present invention may comprise polyclonal antibodies or monoclonal antibodies. Processes of obtaining such antibodies are well known in the related art and the present inventive antibodies may be also prepared using general processes for preparation of known antibodies and/or anti-serums.

The present inventive antibodies are applied to typical liver cancer diagnostic kits. A variety of diagnostic kits are well known, which include, for example, ELISA kit, a simple diagnostic kit disclosed in U.S. Pat. No. 5,728,587 in which antibody is deposited on a strip to observe degree of color development and diagnose liver cancer, micro-array, etc.

The present invention further provides a screening method of compounds for control expression of polynucleotide and/or protein above mentioned, comprising the steps of (a) contacting candidate compounds with cells; (b) comparing gene expression level of the polynucleotide and/or the protein above mentioned in the cells to that of a control; and (c) selecting the candidate compound that alters gene expression level.

The screening method of the present invention uses polynucleotide prepared according to the present invention as a probe, or the present inventive antibodies in order to screen specific materials capable of altering PPIA expression level. More particularly, the screening method of the present invention can screen (i) liver cancer cells of mammals including human beings, or (ii) any materials to alter PPIA expression level by measuring content of mRNA or protein of PPIA contained in transformed cells prepared by insertion of polynucleotide of the present invention into a vector and transformation of the inserted product in a specific host.

Measurement of mRNA or protein content in PPIA may include quantifying mRNA content by extraction of mRNA from liver cells using known methods, for example, by means of RT-PCR and/or typical Northern blot assays. Alternatively, PPIA content can be quantified by extraction of protein from liver cells using known methods, for example, by typical Western blot assays.

Using the screening method can select materials of improving PPIA expression level as liver cancer accelerating materials, and materials of reducing PPIA expression level as materials for inhibiting activation of liver cancer cells.

The transformed cells used in the above screening method can be prepared by the following procedure.

An expression vector containing any one of polynucleotides according to the present invention is produced by, for example, preparing desired DNA fragment and conjugating the DNA fragment to downstream region of a promoter in the proper expression vector. Examples of the expression vector include: extranuclear genes induced from E. coli such as pBR322, pBR325, pUC12, pUC13, etc.; extranuclear genes induced from Bacillus subtilis such as pUB110, pTP5, pC194, etc.; extranuclear genes induced from yeast such as pSH19, pSH15, etc.; bacteriophage such as sterilized λ virus; animal virus such as RNA tumor virus, baculovirus, etc.; and, pA1-11, pXT1, pRc/CMV, pRc/RSV, pcDNAI/Neo, and the like.

The promoter may include any one which is known to be properly applied to host cells used in expression of genes. For example, when the host cell is animal cell, the promoter can comprise SRαpromoter, SV40 promoter, LTR promoter, CMV (cytomegalovirus) promoter, HSV-TK promoter and so on. Among them, CMV promoter and/or SRαpromoter are(is) preferably used. In case of using Escherichia coli as the host cell, examples of the promoter include trp promoter, lac promoter, recA promoter, λPL promoter, 1pp promoter, T7 promoter, etc. If the host cell is Bacillus spp., the promoter is exemplified by SP01 promoter, SP02 promoter, penP promoter and the like. In case of using yeast as the host cell, the promoter may include PH05 promoter, PGK promoter, GAP promoter, ADH promoter, etc. Lastly, if the host cell is insect cell, P10 promoter is preferably used.

If necessary, the expression vector useable in the present invention further contains enhancer, splicing signal, poly A additional signal, selection marker, SV40 replication origin (SV40 ori), etc. The selection marker includes, for example, dihydrofolate reductase (DHFR) gene, ampicillin resistant gene, neomycin resistant gene and so on.

The transformed cell containing any one of polynucleotides according to the present invention can be prepared by transformation of the host cell using the expression vector containing the polynucleotide by means of known methods. Examples of the host cell include Escherichia coli, Bacillus spp., yeast, insect cell, insects, animal cell, etc.

Transformation is performed using general methods dependent on kinds of host cells. For example, Escherichia coli is transformed by a method disclosed in Proceedings of the National Academy of Sciences (Proc. Natl. Acad. Sci. USA), 69^{th} vol., 2110 (1972) or Gene, 17^{th} vol., 107 (1982). Bacillus spp. is transformed by a method disclosed in Molecular & General Genetics, 168^{th} vol., 111 (1979). Yeast is transformed by a method disclosed in Methods in Enzymology, 194^{th} vol., 182 to 187 (1991) or Proc. Natl. Acad. Sci., USA, 75^{th} vol., 1929 (1978). Insect cell and insects are transformed by a method disclosed in Bio/Technology, 6, 47 to 55 (1988). Animal cell is transformed by a method disclosed in Virology, 52^{nd} vol., 456 (1973).

Transformed cells can be cultured using general methods depending on kinds of host cells. For example, when the transformed cell which was formed using Escherichia coli or Bacillus spp. as the host cell is cultured, a liquid type culture medium is preferably used. Such culture medium also preferably contains carbon source, nitrogen source and/or minerals required for growth of the transformed cell. The culture source includes, for example, glucose, dextrin, soluble component, sucrose, etc. The nitrogen source includes, for example, inorganic or organic materials including ammonium salts, nitrates, peptones and/or bean cake. The minerals include, for example, calcium chloride, magnesium chloride, etc. The culture medium may further include yeast extract, vitamins, and/or growth enhancing factors. The culture medium preferably has pH 5 to 8.

The culture medium for the transformed cell which was formed using Escherichia coli as the host cell preferably includes, for example, M9 culture medium containing glucose and casamino acid described in Miller, Journal of Experiments in Molecular Genetics, 431-433, Cold Spring Harbor Laboratory, New York. 1972. The transformed cell is cultured at about 15 to 43° C. for 3 to 24 hours using the host cell, that is, Escherichia coli. If necessary, the culturing process may be performed under air circulation and/or agitation. As the culture medium for incubating the transformed cell which was produced using insect cell or insects as the host cell, a prepared medium comprising Grace's insect medium disclosed in Grace, T. C. C., Nature, 195, 788 (1962), with addition of non-assimilated 10% bovine serum is preferably used. The prepared medium preferably has pH 6.2 to 6.4. The culturing process is performed at about 27° C. for 3 to 5 days, and if necessary, it is also performed under air circulation and/or agitation. The culture medium for incubating the transformed cell which was produced using animal cell as the host cell, may include, for example: MEM culture medium containing 5 to 20% fetal bovine serum disclosed in Science, 122^{nd} vol., 501 (1952); DMEM culture medium disclosed in Virology, 8^{th} vol., 396 (1959); RPMI 1640 culture medium disclosed in The Journal of the American Medical Association, 199^{th} vol., 519 (1967); 199 culture medium disclosed in Proceeding of the Society for the Biological Medicine, 73^{rd} vol., 1 (1950), etc. Such culture medium preferably has pH 6 to 8. The culturing process is performed at about 30 to 40° C. for 15 to 60 hours, and if necessary, it is also performed under air circulation and/or agitation.

As described above, polynucleotide, protein, antibody and transformed cells according to the present invention can be usefully applied to detection of liver cancer by measuring contents of the above materials in biota samples and comparing the measured values to that of control.

Hereinafter, the present invention will be described in detail from the following preferred experimental examples with reference to the accompanying drawings. However, these are intended to illustrate the invention as preferred embodiments of the present invention and do not limit the scope of the present invention.

### Experimental Example 1

### Preparation of Tissue Sample

For experiment of cDNA micro-array, primary HCC tissues and poorly or non-differentiated normal tissues near HCC tissues were obtained from patients with liver cancer at Chonbuk National University Hospital, Korea. Tissue samples were frozen in liquid nitrogen.

### Experimental Example 2

### Analysis of PPIA Expression in Liver Cancer Tissues

In order to identify correlation of PPIA with progression of liver cancer, gene expressions of PPIA were determined for cancer tissues and poorly or non-differentiated tissues near the cancer tissues and were compared with each other by using cDNA micro-array. As a result, there was observed a significant difference of PPIA expressions in the liver cancer tissues. More particularly, for 96.6% of the patients (29/30 persons), RNA expression of PPIA was increased compared to normal liver tissues (see FIG. 1A). In each of liver cancer grades I to IV, three pairs of liver cancer and non-cancer HCC samples were selected for Northern blot assay to determine RNA level. As shown in FIG. 1B, PPIA expression was significantly increased in Edmonson grades III/IV although it was increased even in Edmonson grades I/II, which relate to degree of differentiation of liver cancer.

### Experimental Example 3

### Production of PPIA Recombinant Protein and Antibody

1. Production of PPIA recombinant protein

2 ml of pGEX4T-1 PPIA DNA was introduced and cultured in 50 ml of DH5a competent cell. After IPTG (isopropyl-1-thio-β-galactoside) treatment, the treated material underwent SDS-PAGE electrophoresis to determine whether there was induction of protein. As a result, it was found that PPIA protein was well expressed by IPTG. After determination of solubility of the induced protein, it was identified that most of PPIA protein expressed by IPTG was insoluble while only a part of the protein was soluble.

Mass production of PPIA protein was practically achieved. PPIA protein in this experimental example had GST-tag, thus, was separated using a glutathione sepharose 4 resin column. By means of SDS-PAGE electrophoresis, it was determined whether the column fraction contains PPIA protein. After the electrophoresis, the obtained gel was delivered into PVDF membrane and was subjected to Western blot assay using PPIA monoclonal antibody in order to determine whether the treated fraction contains PPIA protein.

2. Production of Polyclonal Antibody Using PPIA Protein

10 mg of PPIA protein was intraperitoneally injected into each of BALB/C female mice aged at 6 to 8 weeks using a complete adjuvant and, after 2 weeks, 10 mg of PPIA protein per mouse was IP injected using an incomplete adjuvant. After 3 days, a blood sample was collected through eye bleeding to determine antibody titer thereof. When OD value was not more than 1.0 at 490 nm for at least about 1:1000 of the antibody titer, the blood sample was subjected to boosting at a 2 week interval until the antibody titer was increased.

3. Identification of Specificity

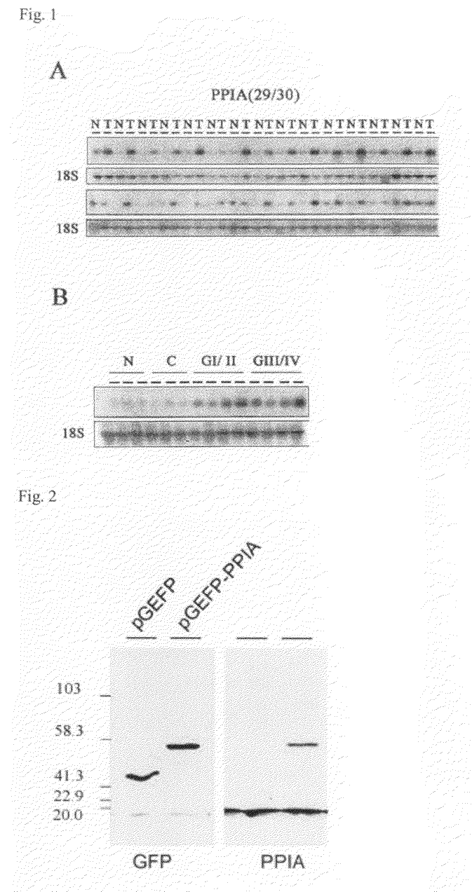

Using GFP marker expression vector instantly expressed recombinant gene of fusion protein and specificity of the gene was observed by means of Western blot assay (see FIG. 2). It was demonstrated that PPIA has the same band with that of GFP fusion protein.

### Experimental Example 4

### PPIA Expression in Supernatant of Liver Cancer Cell Line

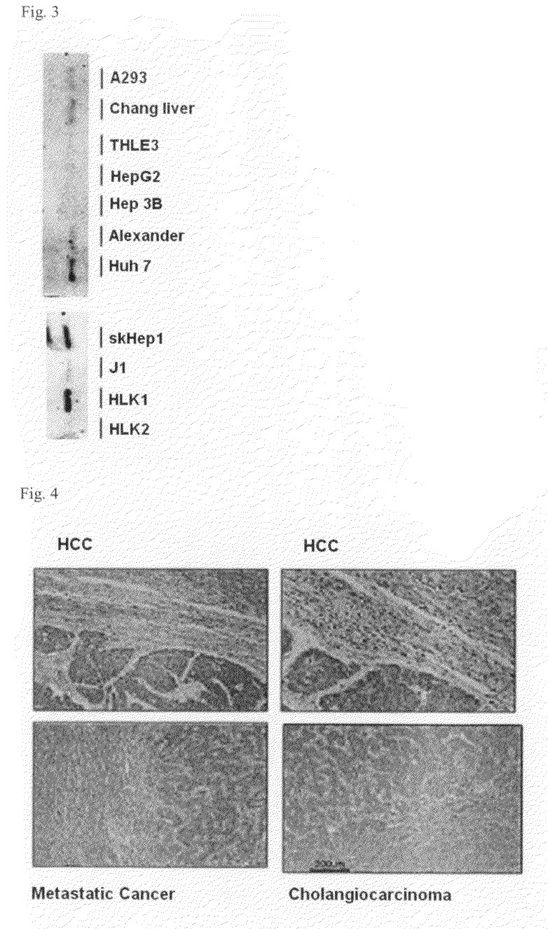

For comparison of protein expressions in liver cancer cell line, Western blot assay was performed by using polyclonal antibody (see FIG. 3). After incubating liver cancer cell line, supernatant of the incubated product was eluted into an elution solution (1% Triton X-100, 150 mM NaCl, 100 mM KCl, 20 mM HEPES (pH 7.9), 10 mM EDTA, 1 mM sodium orthovanadate, 10 mg/ml of aprotinin, 10 mg/ml of leupeptin, 1M PMSF), the cell solution underwent electrophoresis and immunoblotting in a nitrocellulose membrane (Bio-Rad, CA, USA). The immunoblotting membrane was placed in PBS solution containing 5% skimmed milk powder and 0.1% Tween 20, blocking treated at room temperature for 2 hours, reacted using an antibody to PPIA (1:5,000) for 2 hours, and finally reacted using a secondary antibody conjugated with 1:5,000 diluted HRP for 1 hour.

Lastly, the reaction product was developed and analyzed by using a reinforced chemi-luminescent detection kit such as ECL assay kit (Pierce, Ill., USA). As shown in FIG. 3, it was identified that PPIA was abundantly expressed in supernatants of Huh-7, SKHep-1 and/or HLK 1 cell lines. From this result, it was understood that PPIA is one of secretion proteins.

### Experimental Example 5

### PPIA Expression in Liver Tissues by Means of Immunohistochemistry

PPIA protein expression was analyzed in liver tissues which represented clinical and pathological features, by means of immunohistochemistry (see FIG. 4). For this purpose, polyclonal antibody to PPIA (1:1,000) was labeled on a slide by sampling a paraffin-embedded HCC (donor block) centralized tissue biopsy (with diameter of 2 mm) and treating the sample by paraffin removal and antigen retrieval processes. The labeled PPIA was analyzed by means of avidin-biotin complex (ABC) method. Herein, 3,3-diaminobenzidine (DAB) was used as chromogen for the analysis method. A negative control relative to the antibody was saline. If at least 10% of cells in a tissue region are uniformly dyed, the tissue sample is considered positive.

From a result of immunohistochemical analysis, it was found that PPIA expression was increased in metastatic cancer and cholangiocarcinoma tissues and this suggested that increase of PPIA expression is substantially correlated with progression of liver cancer.

### Experimental Example 6

### Determination of PPIA Protein in Serum of a Patient by Means of Immunodot Assay

Polyclonal antibody was used to establish an immunodot assay method and to determine degree of PPIA secretion in serum of a patient. Two kinds of serum samples diluted by 5 times and 10 times (10 individual samples per each case) were prepared. 2 ml of each of the samples was dotted on a nitrocellulose membrane, dried at room temperature and blocking treated with 1% BSAT.

After reaction of polyclonal antibody to PPIA, secondary antibody conjugated HRP was added to the reaction product and the mixture was color developed using DAB.

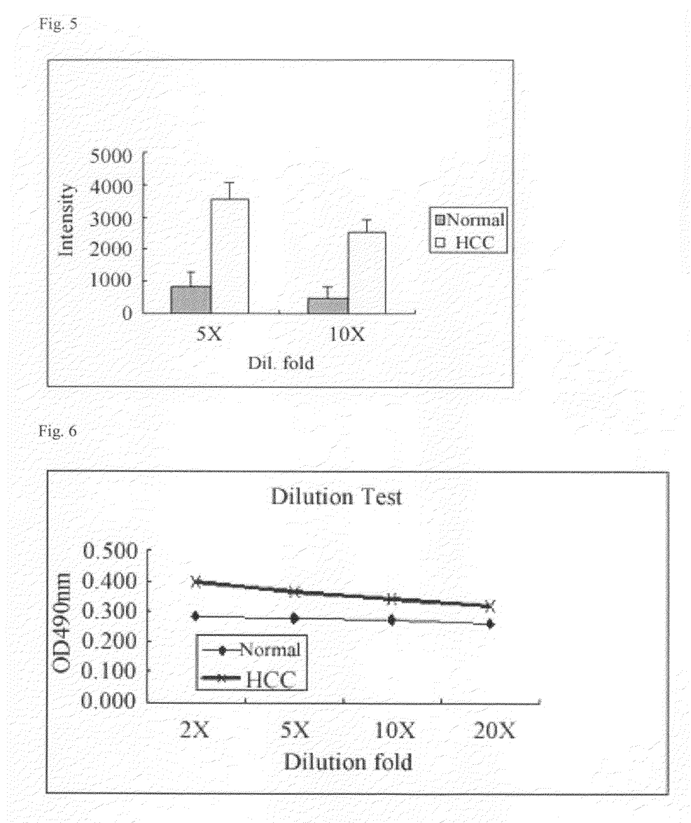

Degree of color development can be scanned and compared between liver cancer tissue and a normal control. From FIG. 5, it was identified that PPIA secretion was greater in liver cancer patients than that of the normal control.

### Experimental Example 7

### Determination of PPIA Protein Concentration in Blood of a Patient by Means of ELISA Assay

ELISA assay was established using monoclonal antibody and polyclonal antibody.

After coating the polyclonal antibody obtained from a rabbit with 1 μg/ml of a sample, the coated antibody was blocking treated using 1% BSAT.

After adding a PPIA sample solution followed by a monoclonal antibody conjugated HRP to the treated antibody, the antibody was developed using TMB (3.3′, 5.5′-tetramethylbenzidine). By ELISA assay, PPIA protein concentration in blood of a patient with liver cancer was determined. As a result of diluting serums of the patient with liver cancer and a normal person and determining ODs (Optical Densities) thereof, PPIA concentration for the patient with the liver cancer was considerably increased, compared to that of the normal person (see FIG. 6). This result indicated that increase of PPIA concentration is closely related with liver cancer incidence.

Consequently, it is understood that PPIA level in liver cancer has high possibility and effectiveness as diagnostic and prognostic factors for liver cancer.

### INDUSTRIAL APPLICABILITY

As described in detail above, the present invention provides PPIA marker for diagnosis of liver cancer, antibody, and a screening method of compounds useful for treatment and/or inhibition of liver cancer.

While the present invention has been described with reference to the accompanying drawings, it will be understood by those skilled in the art that various modifications and variations may be made therein without departing from the scope of the present invention as defined by the appended claims.

1-10. (canceled)

11. A marker for liver cancer diagnosis comprising at least one polynucleotide selected from a group consisting of the following polynucleotides (a) to (d):
(a) polynucleotide having a base sequence defined by SEQ ID NO: 1 or substantially similar sequence to SEQ ID NO: 1;
(b) polynucleotide encoding specific protein composed of an amino acid sequence defined by SEQ ID NO: 2;
(c) polynucleotide that comprises SEQ ID NO: 2, in which at least one amino acid is substituted, deleted, inserted and/or added, and that encodes protein functionally equivalent to specific protein composed of SEQ ID NO: 2; and
(d) polynucleotide that is encoded by another polynucleotide hybridized with polynucleotide having SEQ ID NO: 1 under stringent conditions, and that encodes protein functionally equivalent to specific protein composed of SEQ ID NO: 2.

12. The marker according to claim 11, further comprising alternative polynucleotide encoding partial peptide of protein encoded by the polynucleotide of claim 11.

13. The marker according to claim 11, further comprising protein encoded by a member selected from the group consisting of the polynucleotide of claim 11 and the partial peptide of the protein.

14. An antibody for diagnosis of liver cancer comprising the polynucleotide of claim 11 as an antigen that is conjugated to the polynucleotide, the protein, or a combination of the polynucleotide and the protein.

15. An antibody for diagnosis of liver cancer comprising the protein of claim 13 as antigen that is conjugated to the protein, the polynucleotide, or a combination of the polynucleotide and the protein.

16. A liver cancer diagnosis kit including polynucleotide of claim 11 as a marker for the diagnosis of liver cancer.

17. A liver cancer diagnosis kit comprising the protein of claim 13 as a marker for diagnosis of liver cancer.

18. A liver cancer diagnosis kit including antigen of claim 14.

19. A screening method of compounds for control expression of polynucleotide of claim 11, comprising the steps of:
(a) contacting candidate compounds with cells;
(b) comparing gene expression level of the polynucleotide of claim 11 in the cells to that of a control; and
(c) selecting the candidate compound that alters gene expression level.

20. A screening method of compounds for control expression of protein of claim 13, comprising the steps of:
(a) contacting candidate compounds with cells;
(b) comparing gene expression level of the protein of claim 13 in the cells to that of a control; and
(c) selecting the candidate compound that alters gene expression level.

21. A diagnosis method of liver cancer, comprising the steps of:
(a) measuring content of polynucleotide of claim 11 in a biota sample; and
(b) comparing the measured value to that of a control, in order to detect liver cancer.

22. A diagnosis method of liver cancer, comprising the steps of:
(a) protein of claim 13 in a biota sample; and
(b) comparing the measured value to that of a control, in order to detect liver cancer.

23. A vector comprising the polynucleotide of claim 11.

24. A method of transforming a cell by preparing a transformation of the vector of claim 23 in a host cell.

25. A transformed cell prepared by the method of claim 24.
